(19) United States
(12) Patent Application Publication
(10) Pub. No.: US 2023/0400971 A1
(43) Pub. Date: Dec. 14, 2023

(54) METHOD FOR PROVIDING SERVICE THAT INPUTS AND SHARES ENTITY OBSERVATION INFORMATION, AND COMPUTER-READABLE STORAGE MEDIUM

(71) Applicant: HEALTHCARE BANK CO., LTD., Seoul (KR)

(72) Inventor: Joo Ho TAI, Seoul (KR)

(21) Appl. No.: 18/032,304

(22) PCT Filed: Oct. 13, 2021
(86) PCT No.: PCT/KR2021/014138

§ 371 Date: Apr. 17, 2023

(30) Foreign Application Priority Data
Oct. 16, 2020 (KR) 10-2020-0133935

(51) Int. Cl. G06F 3/04845; G16H 30/40; G06T 19/20; G06T 13/20; G06F 3/04815

(52) U.S. Cl. CPC G06F 3/04845; G16H 30/40; G06T 19/20; G06T 13/20; G06F 3/04815; G06T 2200/24; G06T 2210/41; G06T 2210/61; G06F 2203/04804

(57) ABSTRACT

The present invention relates to a method for providing a service that inputs and shares observation information about a random entity, and a computer-readable storage medium in which commands for executing the method are stored. The method according to the present invention comprises steps of: loading a three-dimensional entity model; providing an interface capable of inputting observation information onto the three-dimensional entity model; and receiving pieces of observation information from a user through the interface, wherein the observation information includes a painting inputted by the user using a marker displayed on the entity model.

### TECHNICAL FIELD

The present invention relates to a method of providing a service for inputting and sharing observation information on an arbitrary entity, and a computer-readable storage medium storing instructions for executing the method.

### BACKGROUND ART

With the rapid development of devices and networks, environments capable of sharing information on various fields and various types are provided, and the demand for such information sharing continuously increases in accordance with technological and industrial development.

On the other hand, there has been a great demand for a system and methodology capable of sharing observation information about an entity, in particular, health care information or medical-related information of an entity, and until recently, various types of systems for sharing various types of information have been proposed.

However, as conventional systems for sharing observation information are not based on graphics, there is a problem in that a user provided with shared information is difficult to understand the observation information about a corresponding entity, and since even the graphic-based information-sharing systems are mostly based on two-dimensional graphics and record the observation information on a paper (paper format), intuitive inputs derived from three-dimensional observations cannot be performed properly for the user, and as a result, it is extraordinarily difficult to accurately understand original intuitive observation information. In addition, as the observation information has not been converted into digital data in a situation of recording on a paper, it is very difficult to store the observation information in a digital form or standardize the data based on the digital form. In addition, although the observation information is based on 3D graphics, there has been an important problem in that a user may not input information as is memorized originally or input of the information is hindered fundamentally, as the memory that relies on the entity information intuitively observed in the initial stage makes the user's attention/surroundings distracted due to the configuration of numerous and various descriptions, steps, and divided boxes.

The present invention is intended to provide an environment that allows intuitive observation of an arbitrary entity and input of initially memorized important information as it is, and also allows easy storage and sharing of the information as valid data. An object of the present invention is to fundamentally exclude the factors that can be refracted, distorted, or changed as the memories of initial observation based on intuitive facts are diversely interfered and obstructed in the conventional process of input or the like, and to conveniently input and effectively store starting from the information that primarily requires the memories and input of the observation or information that is easy to lose, using a vivid and intuitive 3D-based graphic interface that is friendly to the transfer of the initial intuitive memories. In addition, another object is to accumulate 3D-based information input by the user on the interface as diversified and multi-purpose intelligent original data so as to be shared with acquaintances or experts in the neighborhood or utilized in various specialized fields (anatomy, pathology, imaging, medical care, biotechnology, clinical cases, health/epidemiology, climate, agriculture, data science, artificial intelligence). Such data may be effectively used by linking between user classes without difficulty in sharing the data between users, experts, or general users and experts. Furthermore, as the latest artificial intelligence can be utilized through time-series and integrated datasets, in addition to the characteristics of multi-modal and multi-dimensional observation data, systematic and reasonable, especially precise and accurate analysis and prediction can be made. As an example, the object of the invention is to preemptively and successfully carry out early detection and forecast, and timely provision of opinions, responses, services, measures, prevention, and the like in each corresponding disease field through utilization of observation data information collected from incurable chronic diseases and fatal diseases and collected on the basis of the present invention described above in the early stage of spread of recent new infectious diseases.

The present invention has been derived in view of the above problems, and it is possible to solve the technical problems described above, as well as to provide additional technical elements that cannot be easily invented by those skilled in the art.

### DISCLOSURE OF INVENTION

### Technical Problem

Therefore, the present invention has been made in view of the above problems, and it is an object of the present invention to provide a basis of an interface and platform service for a user to conveniently, naturally, and sequentially input truth-based intuitive observation information on arbitrary entities including animal and non-animal entities or important memory images thereof starting from those that are easily influenced and changed in a systematic and reasonable order to increase the value of linking bidirectional data for utilization between general persons, experts, or business partners. Specifically, an object of the invention is to allow a user to input various information on a 3D model while a person, a companion animal, a plant, or an inanimate object is implemented as a 3D model. Particularly, an object of the present invention is to allow a user to input desired information only through intuitive handling on a 3D model.

Another object of the present invention is to intuitively and realistically display abnormalities confirmed by appearance, such as the state and degree of wounds, the degree and feeling of pain, and the like, and convert the abnormalities into digital data so that a user may easily grasp and compare current and past states of a corresponding entity or/and group in time series in the future to make a better insightful determination by recording observation information on the entity in a time-series and cumulative manner and visualizing the information in a summarized form to be shown easily and clearly at a glance.

In addition, another object of the present invention is to display internal components of an entity including organs, tissues, or implants as a 3D model, and easily input desired information on the internal components of the entity so that input of proper information may be possible for both the inside and outside of the entity.

In addition, another object of the present invention is to more easily, realistically, and vividly input, grasp, and compare changes in the observation information about an entity, e.g., changes in the behavior or posture, by inserting animation effects when needed, in addition to implementing a 3D model.

In addition, another object of the present invention is to analyze correlations between abnormal states by utilizing observation information collected from a plurality of user terminals.

In addition, another object of the present invention is to record sounds (crying, barking, and other sounds) made by an entity or generated in the surroundings as a type of observation information so that whether there is an abnormal state can be determined easily on the basis of the sounds.

In addition, another object of the present invention is to provide diversified information observed in time series and/or cumulatively within an entity in a time-series manner on the basis of the location or coordinates of the entity on a 3D model.

In addition, another object of the present invention is to allow a user to record the state of internal organs in detail by utilizing a 3D model and various tools, as well as inputting the state as is felt according to an intuitive and understanding-based flow, by displaying the internal organs or the like on a 3D model and also displaying predicted changes in the organs.

Meanwhile, the technical problems of the present invention are not limited to the technical problems mentioned above, and unmentioned other technical problems will be clearly understood by those skilled in the art from the following descriptions.

### Technical Solution

To accomplish the above objects, according to one aspect of the present invention, there is provided a method of providing a service for inputting and sharing observation information on an arbitrary entity through a user terminal, the method comprising the steps of: loading a three-dimensional entity model; providing an interface capable of inputting the observation information onto the three-dimensional entity model; and receiving the observation information from a user through the interface, wherein the observation information includes a painting input by the user using a marker displayed on the entity model.

In addition, in the method described above, when an arbitrary handling is input by the user, a predetermined animation effect may be executed on the loaded three-dimensional entity model.

In addition, the method may further comprise, before the step of loading a three-dimensional entity model, a pre-check input step of receiving an input of symptom records of the entity from the user.

In addition, the method may further comprise, after the step of receiving the observation information, a step of outputting the received observation information through a display of the user terminal, wherein the observation information output through the display of the user terminal may be displayed based on any one among a time, a location, a specific medicine, and a specific treatment.

In addition, in the method described above, the three-dimensional entity model may further include internal components existing inside the entity model, wherein a degree of display of the internal components may be adjusted by a handling input of the user.

In addition, in the method described above, in the three-dimensional entity model, body transparency may be adjusted by a handling input of the user.

In addition, in the method described above, a marker that can be used when the user inputs the observation information may be displayed on an outer surface of the entity model or an outer surface of internal components of the entity model, wherein the marker may be a three-dimensional shape having an arbitrary volume value.

In addition, in the method described above, the three-dimensional entity model may be obtained by converting 3D scan data obtained through a 3D scanning means provided in the user terminal or a 3D scanning means provided in another terminal.

In addition, in the method described above, the internal components existing inside the entity model may include internal organs of the entity model, and the observation information may include an input of a piercing bullet wound penetrating the organ, an input of a pain associated with a source part of an arbitrary disease, or an input of a pathological change.

In addition, in the method described above, the internal components existing inside the entity model may include a plurality of biological tissues, and the observation information may include a brush input associated with at least two or more biological tissues among the plurality of biological tissues.

On the other hand, a method of providing an observation information sharing service according to another embodiment of the present invention may comprise the steps of: receiving observation information input from a plurality of user terminals; and searching for an association pattern on an entity from the received observation information.

On the other hand, in a computer-readable storage medium that stores instructions for performing a method of providing a service for inputting and sharing observation information on an arbitrary entity according to still another embodiment of the present invention, the method may comprise: the method of providing a service for inputting and sharing observation information on an arbitrary entity includes the steps of: loading a three-dimensional entity model; providing an interface capable of inputting the observation information onto the three-dimensional entity model; and receiving the observation information from a user through the interface, wherein the observation information includes a painting input by the user using a marker displayed on the entity model.

### Advantageous Effects

According to the present invention, there is an effect of allowing a user to intuitively and easily input truth-based observation information about an animal entity or a non-animal entity.

In addition, according to the present invention, as observation information is visualized easily and clearly in time series and shown to a user so that the user may easily grasp and compare changes in an entity over time, there is an effect of deriving accurate predictive power and solution as the user acquires superior insight and clear understanding.

In addition, according to the present invention, as observation information may be input even for the internal components of an entity, there is an effect of easily grasping the states of both the inside and the outside of the entity.

In addition, according to the present invention, since input observation information can be easily shared with other people and accordingly it is helpful and may provide a basis for connecting to a practical and integrated solution even in providing timely opinions and responses such as early detection, notification, forecast, and the like of diseases determined by abnormal symptoms before visiting hospitals and specialized institutions, establishing effective connections with related members and organizations, providing diagnosis, first aid, prescription of veterinarians or medical staff, and providing follow-up procedures and prevention by hospitals and related institutions, there is an advantage of effectively receiving and understanding accurate, precise, and rapid understanding-based methods or guidelines for both diversified and various ordinary people and experts.

In addition, according to the present invention, as observation information collected from a plurality of user terminals can be analyzed, and abnormal states related to each other can be distinguished from the observation information, there is an effect of revealing meaningful correlations or differences between abnormal or characteristic states.

In addition, according to the present invention, since a sound generated by an entity or/and surroundings is also considered as one of observation information and used for analysis, there is an effect that it is helpful in determining existence of an abnormal state in association with the sounds of a corresponding entity or/and surroundings.

Meanwhile, the effects of the present invention are not limited to the effects mentioned above, and unmentioned other effects will be clearly understood by those skilled in the art from the following descriptions.

### BRIEF DESCRIPTION OF THE DRAWINGS

FIG. 1 is a view schematically showing an entire system that can be constructed to implement a method according to the present invention.

FIG. 2 is a view schematically showing a method according to the present invention in order.

FIG. 3 is a view showing a main screen on which an entity model is loaded.

FIG. 4 is a view showing an embodiment in which animation effects are added to an entity model.

FIG. 5 is a view showing an example of an interface that allows selection of an internal component of an entity model, more precisely, an internal organ of the entity model.

FIG. 6 is a view showing tools needed to display observation information about an entity model.

FIG. 7 is a view showing an example of an interface in which a cone-shaped marker is displayed on an internal component of an entity model.

FIG. 8 is a view for explaining how a cone-shaped marker is displayed together with the surface of an entity model, and FIG. 9 is a view for explaining an embodiment in which the cone-shaped marker is extended long.

FIG. 10 is a view for explaining a cylindrical marker.

FIG. 11 is a view for explaining a pre-check step.

FIG. 12 is a view showing input observation information displayed in time series.

FIG. 13 is a view for explaining a process of analyzing a pattern on the basis of observation information collected from a plurality of user terminals.

FIG. 14 is a view for explaining a process of confirming whether an entity is in an abnormal state by recording and analyzing sounds of an entity.

FIG. 15 is a view showing conversion of a three-dimensional entity model into a two-dimensional standardized entity model.

### BEST MODE FOR CARRYING OUT THE INVENTION

Details of the objects and technical configurations of the present invention and operational effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are merely examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software configurations that perform the same function.

In addition, the expressions including certain components are expressions of “open type” and only refer to existence of corresponding components, and should not be construed as excluding additional components.

Furthermore, when a certain component is referred to as being “connected” or “coupled” to another component, it may be directly connected or coupled to another component, but it should be understood that other components may exist in between.

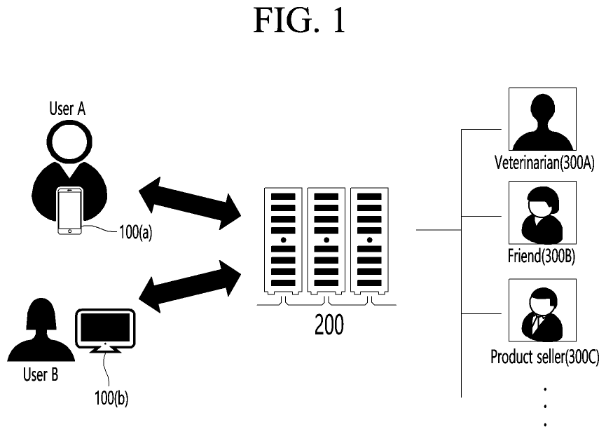

Prior to full-fledged description, first, a system environment for implementing a method of providing a service for inputting and sharing observation information according to the present invention will be described briefly with reference to FIG. 1.

FIG. 1 is a view schematically showing a system that provides a method according to the present invention. In this system, a user terminal 100, a service server 200, and other terminals 300A to C may exist in a network connection state as shown in FIG. 1. Describing an easy example of a method implemented in the present invention, for example, a user who lives together with a companion animal may input observation information about the appearance, pain, symptoms, emotions (mood), photographs, sounds, or observation information on the behavior of his or her companion animal through his/her user terminal 100, and furthermore, may further input observation information about the user himself or herself that can be associated with information on the companion animal. The observation information input in this way may be stored in the service server 200 or a cloud server, and the stored observation information may be shared with others, for example, veterinarians, friends/acquaintances, product sellers, and the like. Meanwhile, the method implemented in the present invention does not necessarily limit an entity to companion animals such as dogs, cats, or the like, and it is understood that the entities mentioned in this detailed description may include other animals, insects, plants, inanimate objects, and the like, as well as human beings. However, it is noted in advance that in the following detailed description, it will be described on the premise that the entity is a companion animal to help understanding of the present invention.

Meanwhile, each of the components constituting the system shown in FIG. 1 will be described in more detail as follows.

First, in relation to the user terminal 100, the user terminal 100 refers to a terminal possessed or carried by a user, and it may include installation-type terminals such as desktop PCs, kiosks, and the like, as well as portable terminals such as smartphones, smart watches, tablet PCs, VR/AR devices such as smart goggles (glasses), and the like. In addition, the user terminal 100 may also include other types of wearable devices such as smart gloves and smart lists, and these wearable devices use a gyro sensor function to make an input based on a method of detecting the posture of the device. When seeing these user terminals from the aspect of a device, it is assumed that each user terminal has a central processing unit (CPU) and a memory. The central processing unit may also be referred to as a controller, a microcontroller, a microprocessor, a microcomputer, or the like. In addition, the central processing unit may be implemented by hardware, firmware, software, or a combination of these, and when the central processing unit is implemented using hardware, it may be configured of application specific integrated circuits (ASIC), digital signal processors (DSP), digital signal processing devices (DSPD), programmable logic devices (PLD), field programmable gate arrays (FPGA), and the like, and when it is implemented using firmware or software, the firmware or software may be configured to include modules, procedures, functions, or the like that perform the functions or operations described above. In addition, the memory may be implemented as read only memory (ROM), random access memory (RAM), erasable programmable read only memory (EPROM), electrically erasable programmable read-only memory (EEPROM), flash memory, static RAM (SRAM), a hard disk drive (HDD), solid state drive (SSD), or the like.

For reference, it will be described in this detailed description assuming that the user terminal 100 is a smart phone or a tablet PC to help understanding of the present invention. In this case, the user terminal 100 may include a display, a touch-sensitive surface, a microphone, and the like, and additionally, one or more other physical user input means such as a physical keyboard, a mouse, and/or a joystick may be further connected. In addition, the user terminal 100 may further include a means for sensing and recording sounds or voices, and a means such as a gyro sensor for sensing postures. Meanwhile, various applications executed on the user terminal may optionally use at least one general-purpose physical user input means such as a touch-sensitive surface. Corresponding information displayed on the user terminal, as well as one or more functions of the touch-sensitive surface, may be optionally adjusted and/or changed from one application to the next application and/or within an individual application. In this way, a general-purpose physical architecture (such as the touch-sensitive surface) of the user terminal may optionally support a variety of applications using user interfaces that are intuitive and clear to the user.

The user terminal 100 allows a user to intuitively and easily input observation information about an entity exactly as it is according to a user interface described below.

Meanwhile, the service server 200 is a program for actually implementing a method according to the present invention, i.e., a configuration that provides a set of instructions, and furthermore, corresponds to a configuration that stores observation information input and uploaded from a plurality of user terminals, and it is also a configuration for providing the observation information to a plurality of other terminals.

The service server 200 may be at least one server PC managed by a specific operator, or may be a cloud server provided by another company, i.e., a cloud server that an operator joined as a member may use. Particularly, when the service server is implemented as a server PC, the service server may include a central processing unit and a memory, and since they have been described in detail in the previous process of describing the user terminal, a description thereof will be omitted here.

The schematic structure of the entire system that can be constructed to implement the method according to the present invention has been described above. Hereinafter, a method according to the present invention, i.e., a method of inputting observation information about an entity and providing a history summary visualization function and a sharing function will be described in detail.

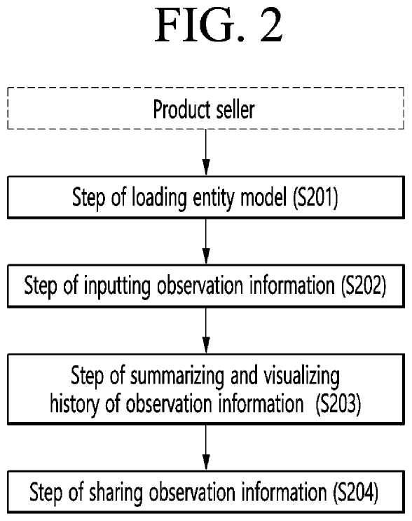

Referring to FIG. 2, a method of providing a service for inputting and sharing observation information according to the present invention may be largely divided into the steps of loading an entity model (S201), inputting observation information (S202), summarizing and visualizing observation information history (S203), and sharing the observation information (S204), and may further execute as needed a step of inputting a pre-check item that is generally previously or selectively performed. Hereinafter, each of the steps will be described.

[Step Loading Entity Model]

In the service providing method according to the present invention, it may begin with the steps of loading an entity model (S201). For example, after a user executes an application and logs in, an entity model previously stored by the user or previously set in the application may be loaded. At this point, the entity model may include an animal entity model (companion animal, human) or a non-animal entity model (plant, inanimate object).

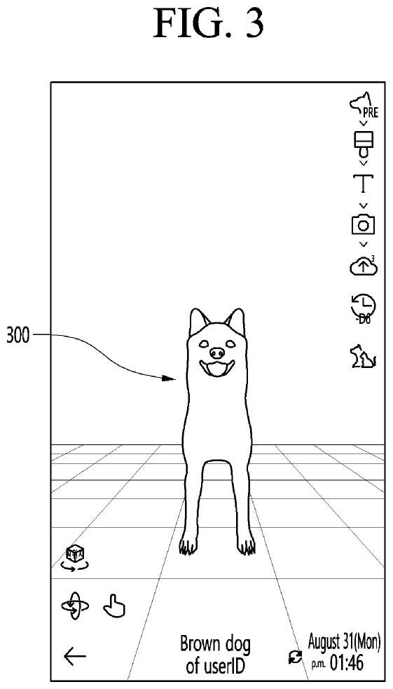

FIG. 3 shows a screen of a user terminal in a state in which the entity model 300 is loaded, and in this detailed description, it will be described assuming that the entity is a dog for convenience. Loading the entity model 300 may be understood as a process the same as loading a modeling file previously stored in a memory provided in the user terminal, and at this point, the modeling file may be downloaded in advance from the service server or created by the user through 3D scanning. In the latter case, the user may directly register the modeling file obtained by 3D scanning in the service server 200 and allow the application installed in the user terminal to download the modeling file, or directly store the modeling file in the memory of the user terminal and allow the application to directly load the modeling file. Particularly, when a 3D scanning means is provided in a portable terminal of the user or in another device of the user, an environment in which the user may directly scan his/her dog three-dimensionally and convert 3D scan data into an entity model that can be loaded on the application can be implemented, and through the process, the user may input more accurate observation information. At this point, the conversion work itself may be supported to be performed on the application.

On the other hand, the user may transfer observation information previously inputted on the basic entity model to be used on a newly created three-dimensional entity model. That is, while using the application according to the present invention for a predetermined period of time, the user may desire to replace the basic entity model with a model of an entity that he or she actually raise, and at this point, and at this point, the user may upload the entity model obtained by 3D scanning on the application and then replace the new entity model with the old entity model so that the entity model that the user actually raises may be output on the application. At this point, the observation information input into the entity model in the past may be matched first and moved onto a standard (predefined) entity model, and then transferred through a process of inputting again the observation information into the new entity model.

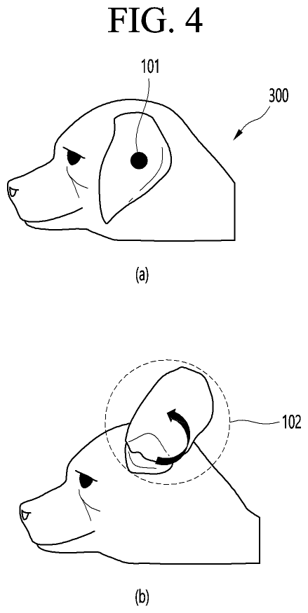

Meanwhile, animation effects may be further added to the entity models loaded on the application. For example, as shown in FIG. 4(a), when the user clicks the animation icon 101 after the animation icon 101 is displayed in a specific part of the entity model, an animation effect may be displayed in the corresponding part as shown in FIG. 4(b). FIG. 4 shows an example of displaying the animation icon 101 on the portion of a dog's ear and showing an animation effect 102 of lifting the ear when the animation icon 101 is clicked. In this way, when an animation effect is added, there is an effect of allowing the user to confirm a part that is difficult to see as it is covered by a specific part through an animation effect, and the user may make a desired input (brush input) on the surface of the entity model revealed after the animation effect is displayed. Meanwhile, the animation effect may be displayed at a time from the beginning to the end of the animation effect by way of one click, or it may be implemented so that only as many animation effects as desired by the user may appear according to the user's handling. That is, the animation effect may be displayed only as much as desired by the user according to the duration of touching a part on the entity model to which the animation effect is added, the number of touches, or the intensity of the touch. In addition, the animation effect may not be necessarily displayed by an act of clicking an icon, and for example, the animation effect may be displayed through a drag input action. In the case of the ear part shown in FIG. 4, the user may drag the dog's right ear so that the ear part is lifted, and then input (brush input, text input) whether there is an abnormality inside the ear. Alternatively, the animation effect may be accomplished based on recognition of touch pen input of the user or the shape (motion) of a hand. For example, an animation effect may be displayed at a specific part of an entity by allowing the user to approach a hand to the ear part of the displayed entity model and having the user terminal recognize a specific motion of the hand. Alternatively, as for the effect, an animation effect may be displayed at the part of the entity by a command “lift the ear” using a voice recognition function. This may be equally applied to the tail, mouth, or the like. In addition, through this animation function, the shapes of an ear according to various emotions or abnormal symptoms (diseases) of a companion animal can be effectively displayed, and the shapes of the arm, leg, and tail may also be implemented to be applied with the animation effect in the same way.

On the other hand, the animation effect as described above has an effect of inputting a symptom that is shown when a specific posture or a specific motion of an entity model is taken. That is, entities may show a tendency of different pains or a tendency of different reactions in various postures such as standing, lying, prone, and kneeling postures. In the service according to the present invention, the posture of an entity model may be created in a form desired by the user, and there is an effect of monitoring more accurately by allowing the observation information to be input onto the entity taking such a posture.

A state in which the entity model 300 is loaded has been described above with reference to FIGS. 3 and 4.

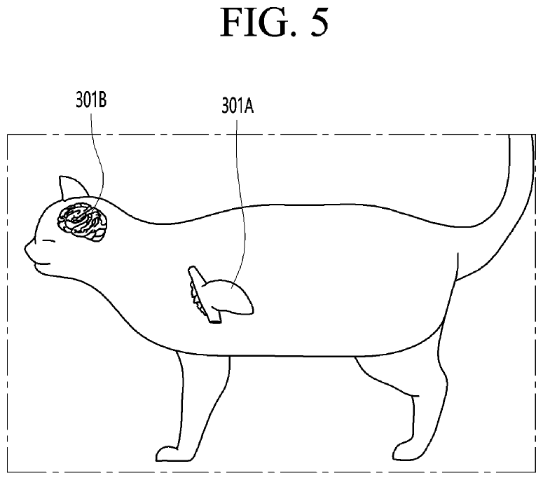

For reference, loading the entity model 300 does not mean that only the external appearance of the entity model 300 is placed in a state capable of accepting any input as shown in FIG. 3 or 4, and may mean that the inside of the entity model 300 is also placed in a state capable of accepting an input. For example, the dog in FIG. 3 may have various kinds of organs inside, and components such as the organs existing inside the entity in this way may also be placed in a state that the user may confirm on the screen, and may be placed in a state capable of enlargement/reduction, brush input, text input, and the like as needed. An example of a screen related thereto is shown in FIG. and referring to this, it can be seen that organs such as the heart 301A, the brain 301B, and the like existing in the body of the entity model 300 are displayed on the screen of the user terminal to be confirmed with eyes. For reference, FIG. 5(b) shows the appearance of the entity model 300 actually implemented in 3D to help understanding of the present invention.

That is, a service that also produces or provides even a three-dimensional model of tissues, organs, cells, and the like existing in the entity model can be performed, and when the cells, tissues, organs, or the like are placed in the body, they may be comparatively displayed by appropriately increasing body transparency, or when the entities are separated from the body, they may be fetched after being selected to be utilized as a separate model. In addition, integrated visualization may be accomplished by appropriately adjusting the transparency of all the entities so that the input status of the body and the inside may be seen at a glance. On the other hand, in some cases, the transparency may be adjusted only for those that a marker of a three-dimensional shape is in contact among the tissues and organs. Although it will be described below, in the application according to the present invention, observation information may be input using a marker of a three-dimensional shape, such as a cone, a cylinder, or the like, and at this point, it may be implemented to identify only the tissues and organs in contact with the volume area of the marker and adjust their transparency.

For reference, the integrated visualization has an effect of providing better insights and providing a decisively meaningful solution by visualizing the entities to be understood at once in an integrated manner, rather than a method containing analog and subjective errors of the past, which finds correlations by comparing the internal and external states of an entity from the viewpoints of experts and general people while separately imagining those observed inside and those observed outside. That is, just like that it is easier to understand an entity when seeing an overall three-dimensional CT or MRI image rather than a two-dimensional image, X-ray, or ultrasound, in the present invention, an entity may be easily grasped by accomplishing the integrated visualization three-dimensionally.

For example, organs such as the heart, kidneys, and the like of a companion animal, and organs provided inside and outside the body such as the eyes or anus of the companion animal may also be the subject of three-dimensional modeling, and in some cases, a user may see the organs by increasing the transparency of the body of the companion animal, or the user may display or input symptoms and pain levels, which will described below, in corresponding organs by separately displaying each organ on the screen three-dimensionally or displaying the cross-sections of the organs. In addition, when it is requested to observe the cells in an entity model more carefully and in detail, a corresponding cell may be displayed as a 3D model so that its unique characteristics can be revealed according to the type and shape of the cell. For example, the shape of a cytoplasm may be differently displayed around the cell nucleus according to the type and shape of each cell, for example, in a three-dimensional method. On the other hand, it is understood that when another internal organ exists inside an external organ, it may be implemented to be able to see the internal organ by increasing the transparency of the external organ. That is, the three-dimensional modeling allows organs in an entity to be examined individually, and may be implemented to perform various editing including transparency control for each of the organs. FIG. 5a shows a view transparently showing the outer skin when organs in the three-dimensional modeling are displayed, and FIGS. 5b and 5c are views showing organs such as the brain and the heart inside by displaying the cross-section of the outer surface of the three-dimensional modeling.

Meanwhile, although it is shown in the drawing that the entity model is a dog or a cat, the entity model in the present invention does not distinguish an animal entity from a non-animal entity as described above, and when the entity model to be loaded is a plant for example, it may be implemented to display the inside of the stem, root, and fruit of the plant on the screen, and when the entity model is an artificial organ or an electronic product (e.g., transplanted organ or the like transplanted as needed), it may be implemented to display the circuit board, wires, and the like inside on the screen. At this point, it may be understood that the electronic product includes all devices including at least one among the circuit board and the wire. In addition, electronic products, metallic products, and the like may also be included in the entity model, and at this point, the electronic products, metallic products, and non-metallic (plastic, silicone) products may also include biomedical devices, such as artificial hearts, hearing aids, artificial joints, implants, artificial heart valves, and the like transplanted in the entity.

[Step of Inputting Observation Information]

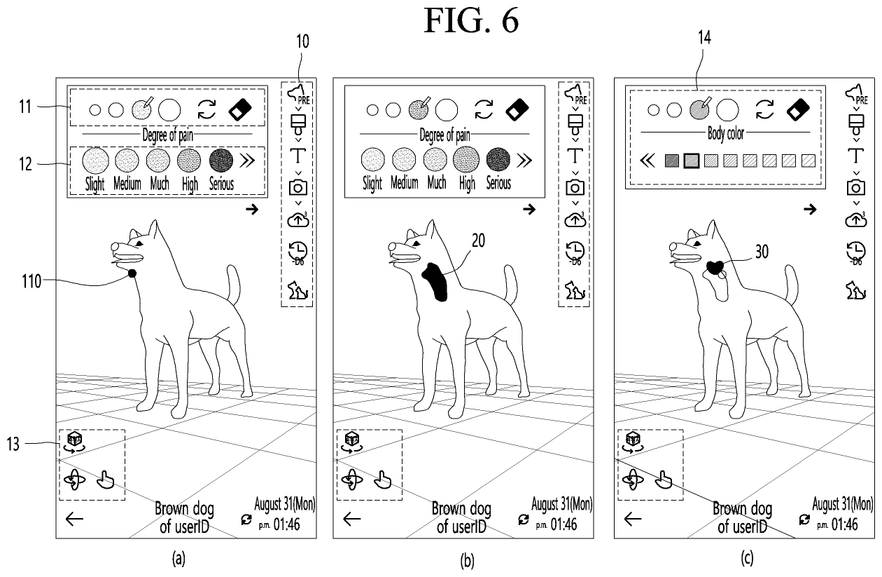

A step of inputting observation information by a user after an entity model is loaded will be described. FIG. 6(a) is a view showing an interface that allows input on the entity model 300, and in addition, FIG. 6(b) is a view showing a state in which a brush input 20 is input on the entity model 300.

Describing the interface briefly, it can be confirmed that the interface includes a plurality of menus 10 for inputting and querying observation information, a selection tool 11 for brush input such as marker size, undo and eraser, a color selection tool 12, a viewpoint change/move tool 13 arranged on the screen. In addition, environmental information (weather, temperature, location, time, date, day of a week) at the timepoint of recording the observation information, the user's name, and the object's name (or title) may be further displayed on the screen.

A user may select an input tool on the interface described above and input desired contents on the entity model 300 or around the entity model 300, and FIG. 6(b) shows an example in which a brush input is made on the surface of the entity model 300 after the user selects the brush tool. Meanwhile, in the interface according to the present invention, a tool for displaying external injuries (bruise, blood, pus) actually generated on the skin surface on the entity model 300 may be independently provided, and the user may separately display invisible pains and visible external injuries using the interface. That is, the interface allows input of invisible pains through brush input as shown in FIG. 6(b) and input of visible injuries through body color input as shown in FIG. 6(c). Although the purpose of allowing input of observation information by distinguishing colors in this way is to allow users to easily recognize information, in addition to this, another purpose is to automatically distinguish and process each detailed information when big data is analyzed for the accumulated observation information in the future.

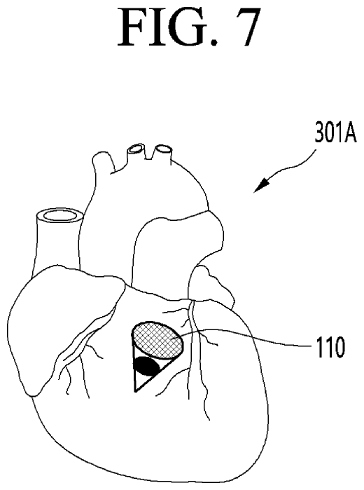

Meanwhile, a marker 110 may be further displayed on the surface of the entity model 300 to apply the brush input on the surface of the entity model 300, and this marker will be described in more detail with reference to FIGS. 7 to 9.

The marker is a tool for accurately pointing an input position, and may be implemented to have a specific geometric shape on the surface of an entity model as shown in FIG. 7. Preferably, the marker may be implemented in a three-dimensional shape, and may be freely moved on the surface of the entity model to accurately inform the user of a desired position for brush input. In addition, the marker may be implemented to move only on the surface of the entity model. That is, as the marker is not allowed to move outside the surface of the entity model, it is convenient for the user to input.

On the other hand, the marker may be used for brush input inside (e.g., an organ) the entity model, as well as on the outer surface of the entity model. For example, companion animals may have arbitrary the same causes of disease in the internal organs, in addition to skin diseases, abrasions, or pain or soreness on the body surface, and it needs to adopt the brush input even in the internal organs so that user's opinions obtained by observation can be reflected. In this case, the marker may be implemented to allow input for the internal organs of previously created three-dimensional entity models so that the user may make a brush input.

FIG. 7 shows an embodiment implementing input of observation information, more precisely, brush input on the surface of the heart 301A, which is one of the internal organs of an entity model, and the heart of the entity may be displayed as a 3D model on the screen of the terminal as shown in the drawing, and a marker 110 is displayed on the 3D model to help the user to easily make a brush input. Since the entity model is three-dimensional, it is desirable that a marker for making any input thereon also has a three-dimensional shape, and although there is no limit on the type of the marker shape, it is assumed that the marker 110 has a conical shape in this description to help understanding. For reference, the marker shape may also be implemented in a spherical shape, and for example, when a user feels pain, although the location of the pain is perceived as being on the body surface, a marker of a spherical shape around the body surface may be used to display the pain in order to easily find the origin of the actual pain. When the cause of a pain is a cancer mass, a parasite mass, constipation, or the like, the marker of a spherical shape (a set of points that exist at a constant distance from the center) makes it possible to guess what the above causes might be, or at least, it may helpful to figure out where to find the cause of the pain in the body.

Meanwhile, cancer masses, parasite masses, constipation inducing factors, or the like that actually cause pain are not necessarily shaped in a regular form such as a sphere, an oval, or the like in many cases, and most of them are atypical. Even in this case, the marker of a three-dimensional shape described above may be useful. For example, a user may mark a point where gastric cancer masses, parasite masses, constipation inducing factors, or the like are expected to exist on the entity model by utilizing the marker of a three-dimensional shape or may input even the shape of a predicted cause of pain by inputting the marker of a three-dimensional shape a plurality of times. In the latter case, in the case of a marker of a spherical shape for example, atypical input may be possible by overlapping some volumes of adjacent marker inputs when the markers are input several times, and this may be equally applied to even a marker of a shape other than the spherical shape.

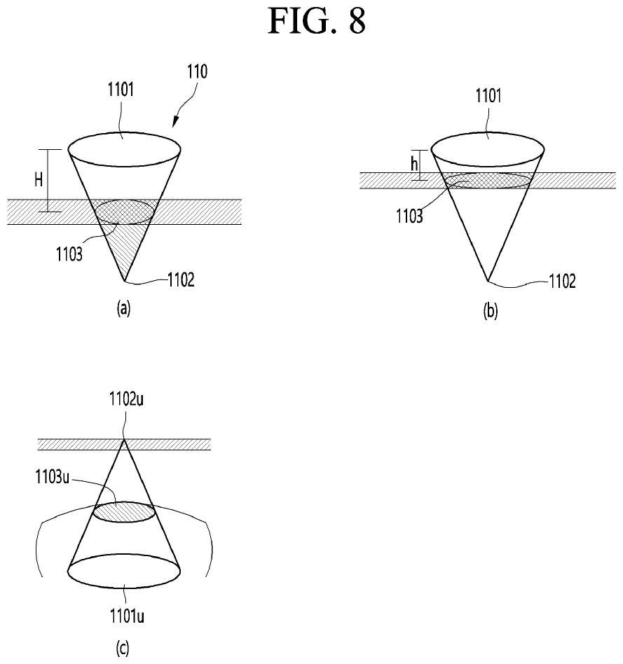

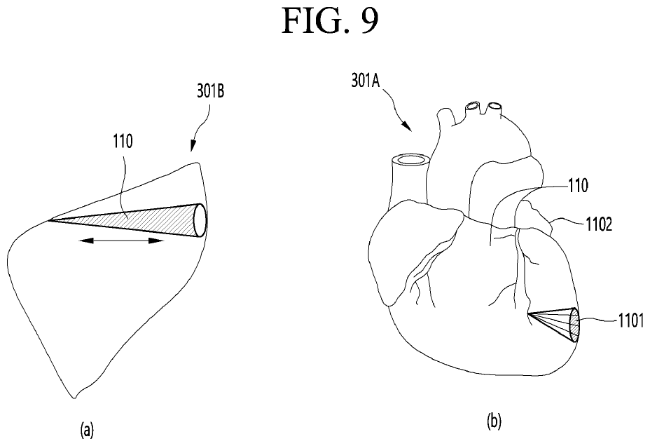

Referring to FIG. 8(a), a marker may be a three-dimensional shape having at least one base and one height, and particularly, a cone-shaped marker may be a three-dimensional shape having a base 1101, a height H, and a tip 1102. Meanwhile, the cone-shaped marker 110 is displayed on the surface of an entity model, and the marker 110 may be implemented to be movable within a range where the surface of the entity always exists between the base 1101 and the tip 1102. This is to enhance user's convenience of handling the marker by preventing the marker 110 not to go out of the surface of the entity. On the other hand, the marker 110 inevitably includes an overlapping surface 1103 overlapped with the surface of the entity model, and the overlapping surface 1103 may be used to allow the user to adjust the size of the input surface. That is, the user may adjust the area of the overlapping surface 1103 by adjusting the height h between the entity surface and the base 1101 as shown in FIG. 8(b). The input area may be adjusted through the adjustment by decreasing the length h between the base 1101 and the entity surface when it is desired to apply a wider range of brush input on the entity surface, and increasing the length h between the base 1101 and the entity surface when it is desired to apply a narrower range of brush input on the entity surface. At this point, when the tip 1102 is adjusted to come into contact with the entity surface, brush input of a point unit will be possible as a matter of course. For reference, adjustment of the area of the overlapping surface 1103 will be possible only in a three-dimensional shape in which the cross-section changes according to the height like a conical shape, and may not be applicable to a three-dimensional shape in which all cross-sections according to the height are the same like a cylindrical shape.

Meanwhile, although only an embodiment in which only one overlapping surface of the marker 110 exists between the base 1101 and the tip 1102 has been described above, two or more overlapping surfaces may exist between the base 1101 and the tip 1102. For example, the base 1101 may be displayed to contact the skin surface of the three-dimensional entity model, and the overlapping surface 1103 may be displayed to contact the heart among internal organs, and on the contrary, the base 1101 may be displayed to contact the internal organs, and the overlapping surface 1103 may be displayed to contact the skin surface of the entity model. As will be described below, the height of the marker 110 may be adjusted, and in this case, the number of overlapping surfaces may be larger than two according to the number of skins and organs through which the marker 110 passes. When a plurality of overlapping surfaces originating from one marker 110 can be displayed in this way, observation information displayed by the overlapping surfaces, base, and tip may be displayed to be associated with each other, and as the pain may be displayed in an arbitrary area of the skin surface and also displayed at a specific internal organ that the marker 110 is in contact, a user may record the relation between external pains and internal organs so that a specific organ may be examined first in case of finding a cause of the pain later.

FIG. 8(c) shows an embodiment in which the overlapping surface 1103u of the marker 110 is in contact with an internal organ, the tip 1102u is in contact with the skin surface of the entity, and the base 1101u exists inside the internal organ. As is shown, the marker 110 may be freely utilized in the interface according to the present invention in accordance with the intention of the user, and particularly, observation information about the entity may be input by controlling the cross-section and the tip of the marker to be in contact with the skin surface, internal organs, and the like of the entity.

Meanwhile, one of the methods of inputting observation information may include a method of allowing a user to put a desired color in an inner area of an outline drawn by a user using the marker. In addition, at this point, as it is further allowed to input a plurality of different colors in the inner area of the outline, the degree of pain and the depth of location where the pain is estimated to exist may be expressed.

For reference, as for the cone-shaped marker, the cone-shaped marker may also be used to probabilistically infer a point where the cause of a pain is located from a specific location specified by the user. For example, when the user makes an input by placing a point where the user feels pain or a point where a dog is licking in pain at the vertex of the cone-shaped marker, the area of the volume occupied between the vertex and the base can be predicted to have a point where the cause of a pain is located with a higher probability than that of an outer area.

In addition, in a general situation, it is expected that there will be an input by the user in a state in which only the outer surface of an entity model can be seen, i.e., a state in which the internal organs cannot be seen, and when an expert such as a veterinarian, a doctor, or the like confirms later that observation information has been input by the marker of a three-dimensional shape, it will be helpful for the expert to find out a cause of the pain more easily as the internal components (organs) of the entity model are shown with adjusted transparency.

Meanwhile, FIG. 9(a) shows an embodiment in which the height of the marker 110 itself can be adjusted, and the interface may allow a user to change the shape itself of the marker 110 as needed by providing the user with a means for adjusting the height of the marker 110 or the width of the base. In addition, the interface may provide an input surface changing means so that input through the marker 110 can be made by the side surface other than the base 1101 or the overlapping surface 1103. For example, FIG. 9(a) shows an embodiment of displaying a piercing bullet wound in the liver 301C of an entity. At this point, the user may adjust the height (length) of the marker 110 and the area of the base to mark the piercing bullet wound, and input a mark of the piercing bullet wound as shown in FIG. 9(a) by changing the input surface to the side surface. On the other hand, the cone-shaped marker 110 may be provided with an interface to display the inlet and outlet of the piercing bullet wound, and the mark of the piercing bullet wound may be displayed even when the bottom and the height are not perpendicular to each other. In the case of a piercing bullet wound, the path of the bullet may be slightly changed in the body by internal organs or bones, and even the refractive property of the piercing bullet wound may be displayed on the interface according to the present invention. In this case, the interface may display a line representing the height of the mark of the piercing bullet wound, and allows the user to drag an arbitrary point on the line representing the height so that the mark of the piercing bullet wound may be refracted. Input of the piercing bullet wound in this way may also be used for a simulation that predicts the direction and appearance of the bullet or fragments, the shape of internal organ damage according to the strength or shape of the penetrated tissue, the degree of internal organ damage, the direction of damage, and the like.

For reference, FIG. 9(b) shows an embodiment in which the cone-shaped marker 110 is displayed in a way that the tip 1102 is in contact with the coronary artery on the surface of the heart 301A, and the base 1101 is in contact with necrotic tissues generated in the heart 301A. FIG. 9(b) shows an example of utilizing the marker 110 when it needs to predict an area where necrosis occurs, for example, when necrosis of tissues in the heart occurs due to blood clots generated in the coronary artery of the heart, and shows an embodiment in which the marker 110 is placed so that the tip 1102 of the marker 110 is in contact with the thrombotic portion of the coronary artery so that the area of the base 1101 extended from the tip 1102 may be predicted to have necrosis. When a problem is discovered at a specific point as described above, the marker 110 in the interface according to the present invention can be used to predict another problem area caused by the problem. Observation information input using the marker 110 on the internal organ may be used to simulate the three-dimensional range of tissue damage around the organ.

According to the present invention as described above, input of a piercing bullet wound penetrating the organ as shown in FIG. 9(a), input of a pain associated with a part causing an arbitrary disease as shown in FIG. 9 (b), and other inputs for clinical symptoms or pathological changes may be recorded and displayed on the entity model, particularly, in the internal organs.

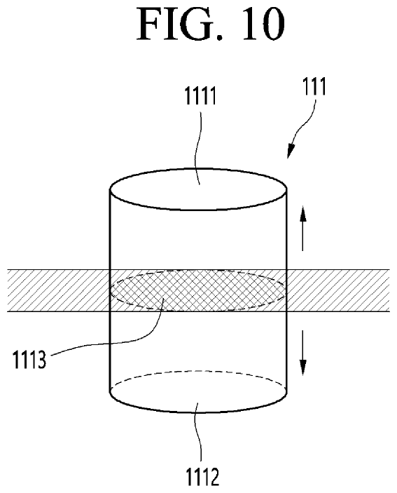

FIG. 10 shows an embodiment when the marker 111 is a cylindrical shape. When the marker is a cylindrical shape, the overlapping surface of the marker 111 may be implemented to allow color input while moving on the surface of the entity model. At this point, the marker 111 may move only within a range allowing the surface of the entity model to exist between the top surface 1111 and the bottom surface 1112 so that the marker 111 may not go out of the entity model.

On the other hand, when the marker 111 is a cylindrical shape, the portion from the base 1112 to a predetermined height of the cylinder (hereinafter, referred to as the lower part of the marker) is displayed on the skin surface of the entity model so that brush input may be performed even inside of the entity model. At this point, the height of the lower part of the marker may be adjusted again to be able to display even the deep inside of the skin of the entity. At this point, brush input at each depth may be performed in different colors. For example, a different pain level may be input according to the depth by repeating the process of extending the lower part of the marker only as much as 1 Cm under the skin and inputting yellow color or a color in the yellow color family at that point, extending the lower part of the marker only as much as 1.5 Cm under the skin and inputting orange color or a color in the orange color family at that point, extending the lower part of the marker only as much as 2 Cm under the skin and inputting red color or a color in the red color family at that point, and the like. Through this process, input of the observation information may be accomplished in an integrated manner throughout the [skin surface—internal organs—deep organs—tissues, cells] of the entity. Of course, the color families described above may be slightly adjusted in the digital processing to have different brightness or saturation to be distinguished from the color of blood, secretions, or wounds found on the actual body surface.

On the other hand, the marker may be converted from a cylindrical shape to a conical shape, or conversely, from a conical shape to a cylindrical shape. A menu (of a toggle type or sliding type) may be separately provided in the interface for conversion of the shape, and a menu (of a sliding type) may be displayed in the interface so that the height of the marker of a conical shape and the marker of a cylindrical shape can be adjusted. Meanwhile, when there is information on the photographs observed using an endoscope or a microscope for biopsy, more accurate observation information on a corresponding entity may be created by displaying the information on the photographs on the entity, instead of the brush input using a marker. At this point, as a method of displaying the photograph information on the entity, for example, a method of converting the photograph information into one layer and then overlaying the layer on a three-dimensional entity model may be used. In addition, an entity may be identified from the photograph information through extraction of a closed curve, and the entity identified in this way is matched on the three-dimensional entity model so that the photograph information may be displayed on the entity model more accurately.

A marker, which is a means necessary for inputting observation information, especially for brush input, has been described above.

Meanwhile, input of the observation information described above may be performed to display the degree of pain or external injury on the outside of the three-dimensional entity model proactively, and display the degree of pain on the internal organ corresponding to the pain or external injury displayed on the outside or input text arbitrarily written by the user subsequently. That is, in the present invention, it may be induced to input observation information in order from the outside to the inside of the three-dimensional entity model, and through this process, observation information from the outside to the inside input by the user may be matched as one related input. Observation information input in this way may be used to reveal the relationship between external injuries or pains and internal organs when big data is analyzed later.

On the other hand, before the step of inputting observation information, there may be a pre-check input step capable of inputting prior information about the entity. For example, as a simple information input interface is provided before or after the three-dimensional entity model is loaded after the user logs in the application, user's convenience can be enhanced. An arbitrary two-dimensional entity model may be displayed before the three-dimensional entity model is loaded, and an interface may be provided to allow the information to be briefly input on the three-dimensional entity model after the three-dimensional entity model is loaded.

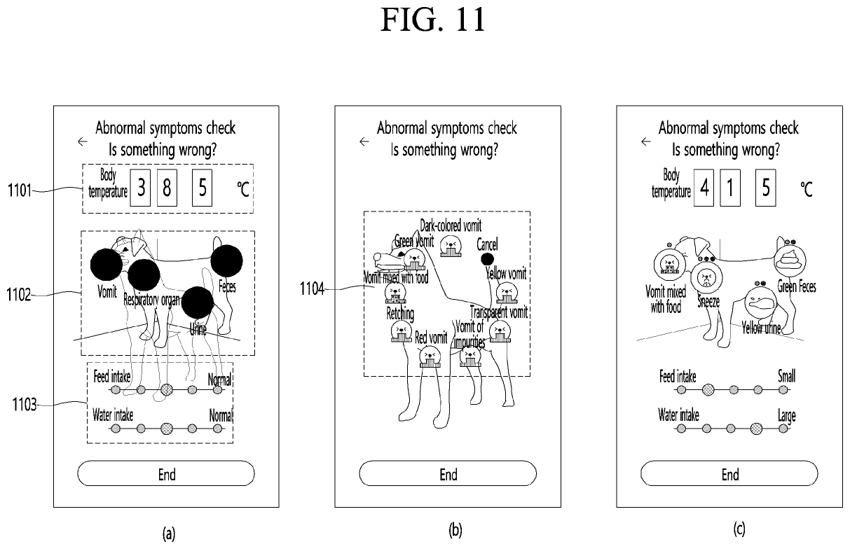

FIG. 11 shows interface screens for the pre-check input step, and the characteristic of the step is not to increase dislike or fatigue of a user even in the periodic repetition of using an application, by accurately completing all the inputs in order while making the input more fun and simpler, as the user may normally use a photograph such as an icon that is clear to understand the essential check parts as much as possible. To this end, the service providing method according to the present invention may include a step of providing main selection options related to an entity (FIG. 11(a)), and a step of providing sub-selection options subordinate to any one main selection option selected by the user (FIG. 11(b)). FIG. 11 shows an interface for inputting a pre-check for a companion animal, and first of all, it can be confirmed that body temperature 1101 and/or weight, vomit, respiratory organ, urine, feces 1102, water, feed 1103, and the like are displayed in FIG. 11(a) as the main selection options related to the companion animal. The main selection options may be configured of items having the highest input frequency or input amount in relation to a corresponding entity, and these main selection options may be selected on the basis of a result of big data analysis performed on the observation information collected by the service server from a plurality of terminals. As needed, the main selection options may be periodically updated to reflect the trend in the observation information over time. For example, during a period when a specific infectious disease is prevalent, there may be a rapid increase in the observation information containing the parts and symptoms related to the specific infectious disease, and the service server may configure the main selection options in a custom-tailored fashion by reflecting the observation information, so that convenience of users in performing the pre-check input can be enhanced. Particularly, in the case of input icons that allow direct input on an entity shape as shown by reference numeral 1102, the position and name of the input icons may vary according to the intention of the service provider. For example, input icons currently configured of vomit, respiratory organ, urine, and feces may be changed to mouth, skin, front legs, hind legs, or hooves (example of foot-and-mouth disease). Next, FIG. 11(b) show a view of displaying sub-selection options subordinate to vomit, when a specific main selection option, e.g., “vomit”, is selected. The sub-selection options may include a vomit color, contents, and other symptoms accompanied with the vomit, and these sub-selection options will vary according to the main selection option. For reference, the sub-selection options may be intuitively selected by the user by displaying the sub-selection options around the main selection option previously selected by the user, and it can be confirmed that the sub-selection options listed by classifying several types of vomits around “vomit” are circularly arranged in FIG. 11(b). Finally, FIG. 11(c) shows the interface after the pre-check input is completed. A noteworthy point in FIG. 11(c) is that one to three small dots of different colors may be displayed on the input icons of reference numeral 1102 in order to easily recognize the degree. This may be an expression showing a difference as much as the bar shape.

It is general that the pre-check input step may be preferably implemented to be executed immediately after the user logs in the application, and the content of the pre-check input may be referenced in determining how to configure an interface (interface for inputting observation information) displayed at a later step. That is, a different interface may be subsequently provided to the user according to the pre-check input content. However, the pre-check input step may not be necessarily executed immediately after login as mentioned above briefly, and it is understood that the interface conversion may be performed so that the pre-check input step may be performed at any time after the entity model is loaded.

The step of inputting observation information and the step of inputting a pre-check have been described above.

[Step of Summarizing and Visualizing History of Observation Information]

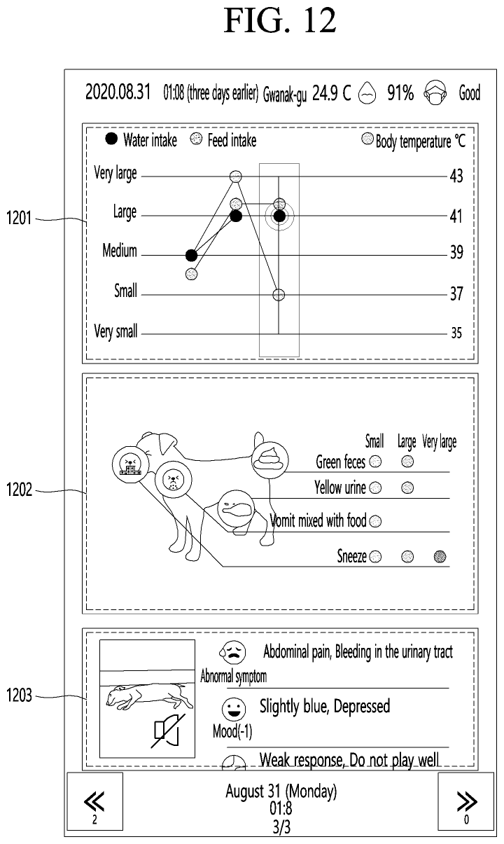

Meanwhile, the observation information input through the interface described above may be cumulatively stored by time (or place), and the accumulated observation information may be displayed in time series (or selectively for a specific place) as shown in FIG. 12.

In the upper part 1201 of FIG. 12, water intake and feed intake input by the user in each date are displayed in the form of a broken line graph. That is, the user may input a specific numerical value by date in relation to an entity, and the cumulatively input numerical values are displayed in the form of a graph as shown in FIG. 12 so that the user may easily and clearly understand the degree of change at a glance. The types of observation information that can be displayed in the form of a graph may further include body temperature, weight, and the like, in addition to the water intake and feed intake.

In the middle part 1202 of FIG. 12, brief information on the entity may be displayed, and it may be implemented to display the observation information input at the pre-check step or the observation information displayed on the three-dimensional entity model. On the other hand, the observation information in the middle part 1202 may be displayed as observation information of another date according to a user's input. For example, as a bar-shaped input icon or a circular input icon is provided and the user is allowed to perform a drag input on the input icon, it is possible to easily and clearly distinguish how the observation information changes by date as much as possible. For example, when a skin disease has occurred on the skin surface of a three-dimensional entity model and the affected area and its degree have been displayed by date from the date of occurrence, the user can easily confirm how the affected area changes through a drag input.

In the lower part 1203 of FIG. 12, at least one among a photograph (or video) capturing an entity, a recording of a sound produced by the entity, or text or an icon input by a user for the entity may be displayed. At this time, the photograph of the entity may be implemented to be enlarged, and the sound recording content may also be implemented to be reproduced.

Meanwhile, although not shown in FIG. 12, an entity model in a brush input state may also be displayed in time series as a matter of course.

As described, the observation information input through the interface according to the present invention may be output on the screen in a summarized and visualized form so that the user may easily and clearly distinguish and recognize the characteristic points at a glance, and particularly, a degree of change in the numerical value and a degree of improvement in pain may be displayed in time series.

Meanwhile, the observation information input through the interface is not implemented to be displayed only in time series, and as needs, it may be implemented to separately display only the observation information input at a specific place, separately display only the observation information when a specific medicine is taken/prescribed, or separately display only the observation information when specific treatments are given. That is, cumulative observation information input by the user may be sorted on the basis of time, location, specific medicine, or specific treatment, and the sorted information may be separately displayed for the user according to each criterion.

[Step of Sharing Observation Information]

Meanwhile, the observation information or pre-check information input as described above may be shared with other users.

In the service providing method according to the present invention, as a user may create a database in the service server 200 by inputting, storing, and uploading observation information (or pre-check information) of an entity, and the service server 200 may provide an environment for the user to share various opinions about the entity by sharing the observation information with other users, and furthermore, allow experts in related fields to use a lot of observation information for education, research, development, commercialization, prevention, and health policies. At this point, sharing the observation information means that the service server 200 permits access of restricted or unrestricted other users when the users give consent to the intent of disclosing the observation information input by the users to others, and such an access permission may be made in a variety of forms, for example, in the form of allowing other users logged in the service server 200 to directly search for corresponding observation information by themselves, or periodically providing updated observation information when mutual permission is given to users who pay for the service of database collection in advance (for example, companion animal observation information of a patient's guardian to a veterinarian in charge).

Meanwhile, numerous observational information uploaded to the service server 200 may be the subject of big data analysis. For example, when input information on the body parts and symptoms of a companion animal included in the observation information, (text) input of a prescription made for the companion animal, input of text and photographs (video) indicating that the symptoms have been improved as days pass, and the like are subjects of big data analysis, there is an effect of obtaining, from the information, a clue as to what prescription or treatment regimen has been effective for the disease or external injury that has occurred in the companion animal. Particularly, when the observation information is collected globally and used as a source of big data analysis, an environment for publicly disclosing and more objectively and scientifically evaluating, determining, and recommending the treatments, folk remedies, or behavioral psychological corrections, which have not been formally dealt with in the field of veterinary, as well as in the field of oriental medicine, medical and health sciences, and related fields of behavioral psychology, may be provided. Machine learning and artificial intelligence (AI) techniques may be applied to the big data analysis, and the big data analysis may include a process of identifying symptoms of a companion animal from the observation information input by the user, a process of tracking the cause of improving each symptom (prescription used for treatment, folk remedy used for treatment), a process of determining whether the cause has a causal relationship in the improvement of symptoms of the companion animal (when it is shown that a specific prescription or a specific folk remedy has been effective in improving the symptoms at a rate of meaningful numerical values, it is determined that there is a causal relationship), and the like. This may be equally applied to a user corresponding to the guardian of a companion animal, i.e., to a person, and regardless of whether the treatments or prescriptions of other entities have been used intentionally or unintentionally for a human or a companion animal, it is expected to contribute to discovery of effective treatments for humans or companion animals. In addition, when these technical concepts and approaches are applied to prevention of new infectious diseases, it is expected to contribute to meaningful integrated early core detection of infectious diseases so that successful early quarantine and response can be effectively accomplished by informatization and insightful visualization of these digital clues themselves and selected clue groups connected to each other more early, quickly, accurately, and precisely, while considering and analyzing useful initial information, such as starting entities, groups, related mechanisms, and the like that are the source of outbreaks of infectious diseases, together with various environmental changes (weather).

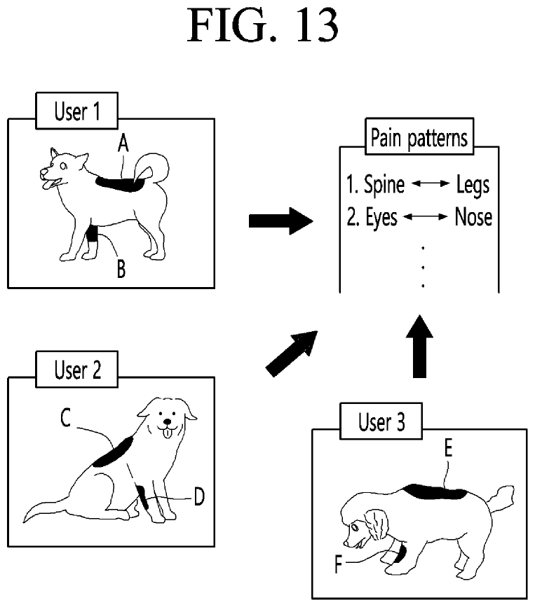

As part of big data analysis, observation information collected through various channels may also be used to find patterns among input items. FIG. 13 schematically shows a process of deriving pain pattern information on the basis of observation information of a companion animal collected from a plurality of users. According to this, in the similar cases where it is grasped that the back of a companion animal (reference numerals A, C, E) and the part between a forefoot and a forefoot joint of the companion animal (reference numerals B, D, F) are marked together in a number of observation information, the service server 200 may infer that the pain in the back and the parts between the forefoots and the forefoot joints of the companion animal tends to appear together in this case, and this pattern of pain may be recorded in the service server 200 or in other databases and shared with users later.

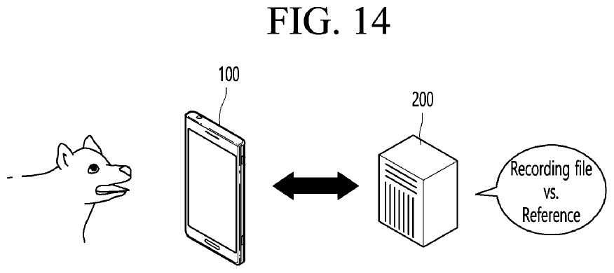

Meanwhile, as part of big data analysis, a sound produced by an entity may also be analyzed and referenced for configuration of reference data related to the sound of the entity, and the reference data may be used in the future to determine a state of the entity when the sound of the entity included in the observation information is input. FIG. 14 briefly shows an embodiment in which a barking sound of a companion animal is input as part of the observation information and shared with the service server 200, and the sound recorded in this way may be compared and analyzed together with reference data in the service server 200. The reference data may exist in plurality according to a situation, and in the case of an example of a companion animal, a sound made by the companion animal according to a disease, a barking sound according to various situations, and the like exist as reference data and may be compared with the sound included in the observation information. Since the reference data may be made on the basis of a large number of collected observation information (sounds), and each of the collected observation information will include recorded sounds together with the situation and state of the entity, the service server 200 may construct the reference data according to each situation.

Meanwhile, such reference data can be a training data set in an AI model analysis, and data used in a test for analysis may be referred to as testing/test data or testing/test data set. Among the sounds produced by an entity, those having similar patterns may be grouped and categorized through AI analysis and big data analysis. By distinguishing the sounds of the entities showing the similar patterns, the sounds may be used as another reference data, or additional analysis may be performed on the sounds themselves of the entities of each grouped pattern.

The step of sharing observation information has been described above.

[Step of Converting Observation Information Input on a Three-Dimensional Entity Model into Observation Information on a Two-Dimensional Entity Model]

In the above description, an embodiment in which various inputs are possible while an entity model is three-dimensionally modeled has been described.

Meanwhile, the method according to the present invention may convert information input on a three-dimensional entity model and store as two-dimensional information or store and use the information as data for analysis. Although complex image data implemented three-dimensionally, i.e., 3D stereoscopic coordinate data, is acceptable for data processing performed on an image, when the complex image data can be converted into simple image data configured on a two-dimensional plane, it will be advantageous much more from the aspect of work efficiency. Particularly, processing two-dimensional image data may be greatly advantageous from the aspect of data processing speed, and focusing on this point, it is intended in the present invention to improve the performance from the aspect of data processing and the efficiency and processing speed of analysis by allowing conversion of data initially input into the three-dimensional entity model by the user as an input on the two-dimensional entity model. In the case of converting into two-dimensional image data and cumulatively storing the observation information, it is expected that the efficiency will be improved greatly, particularly in performing big data analysis and application of an AI model. In addition, as will be described below, it is implemented to convert data into a standard two-dimensional model of a predetermined specification in the present invention, and in this case, since it is easy to compare numerous different types of individuals or species with a standardized model, it is expected that various discoveries can be made from the observation information that is difficult to grasp in the past.

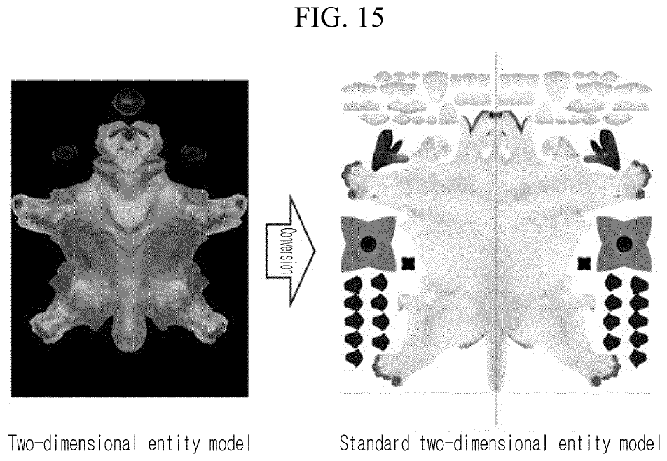

FIG. 15 is a view showing conversion of a three-dimensional entity model into a two-dimensional standardized entity model. When a two-dimensional entity model is displayed on a plane, it may be divided into several parts such as the body, eyes, nose, and the like of an entity as shown in FIG. 15. In addition, brush input on a three-dimensional entity model may be displayed at the same location, more accurately, at a corresponding location, on a two-dimensional entity model. For the conversion into a two-dimensional entity model, it should be assumed that the coordinates on the surface of the three-dimensional entity model correspond to the coordinates of the two-dimensional entity model, respectively. In addition, it is assumed that the eyes and nose of the three-dimensional entity model also correspond to the coordinates of the eyes and nose arranged on the plane of the two-dimensional entity model.

In addition, as shown in FIG. 15, in the method provided by the present invention, as a three-dimensional entity model is converted into a two-dimensional entity model of a standard specification (standard two-dimensional entity model), even different types of species may be displayed as a two-dimensional entity model of the same, at least a similar, specification. For example, in the case of a companion dog, as the size and length of body parts (e.g., legs) are very diverse, when inputs of entity models for several dogs are received from various users, two-dimensional entity models of different sizes may be collected. In this case, the efforts made to reduce the burden of data processing by converting images into two dimensions may not have a great effect. Since conversion into the standard two-dimensional entity model is performed in the present invention to solve this problem, although observation information on two-dimensional entity models of different sizes is collected, data processing such as image comparison or the like for several entity models can be performed easily.

The standard two-dimensional entity model means a two-dimensional model in which a plurality of parameters for displaying the entity model is pre-determined, and it means a standardized body decomposition map model in which various parameters determined in advance, for example, the position of an axis is fixed, and the arrangement position of each part (eyes, nose, teeth, claws) constituting the entity is determined in advance. Meanwhile, when a small-sized entity model is converted into a standard two-dimensional entity model, the conversion can be made in a size closest to the standard two-dimensional entity model by expanding the size of the entity or increasing the length of each part of the entity. However, in this case, it is desirable to maintain the ratios of the entity (e.g., the ratio of the length of the leg to the trunk, the ratio of the length of the nose to the trunk). When conversion to a standard two-dimensional entity model is actively utilized in this way, although observation information is collected for entities of different body sizes, the burden of data processing for mutual comparison is reduced, so that more new information can be obtained from big data analysis on the observation information. In addition, in animal species having a uniquely special structure, for example, an elephant's trunk, a camel's one-humped or two-humped structure, and a legless snake, a standard two-dimensional entity model corresponding to each species may be required.

A method of providing a service for inputting and sharing observation information about an entity and a storage medium for executing the method have been described above. Meanwhile, the present invention is not limited to the specific embodiments and applications described above, and various modifications can be made by those skilled in the art without departing from the gist of the present invention claimed in the claims, and these modified embodiments should not be understood separately from the technical spirit or perspective of the present invention.

1. A method of providing a service for inputting and sharing observation information on an arbitrary entity through a user terminal, the method comprising the steps of:
loading a three-dimensional entity model;
providing an interface capable of inputting the observation information onto the three-dimensional entity model; and
receiving the observation information from a user through the interface, wherein
the observation information includes a painting input by the user using a marker displayed on the entity model.

2. The method according to claim 1, wherein when an arbitrary handling is input by the user, a predetermined animation effect is executed on the loaded three-dimensional entity model.

3. The method according to claim 1, further comprising, before the step of loading a three-dimensional entity model, a pre-check input step of receiving an input of symptom records of the entity from the user.

4. The method according to claim 1, further comprising, after the step of receiving the observation information, a step of outputting the received observation information through a display of the user terminal, wherein the observation information output through the display of the user terminal is displayed based on any one among a time, a location, a specific medicine, and a specific treatment.

5. The method according to claim 1, wherein the three-dimensional entity model further includes internal components existing inside the entity model, wherein a degree of display of the internal components is adjusted by a handling input of the user.

6. The method according to claim 1, wherein in the three-dimensional entity model, body transparency is adjusted by a handling input of the user.

7. The method according to claim 1, wherein a marker that can be used when the user inputs the observation information is displayed on an outer surface of the entity model or an outer surface of internal components of the entity model, wherein the marker is a three-dimensional shape having an arbitrary volume value.

8. The method according to claim 1, wherein the three-dimensional entity model is obtained by converting 3D scan data obtained through a 3D scanning means provided in the user terminal or a 3D scanning means provided in another terminal.

9. The method according to claim 5, wherein the internal components existing inside the entity model include internal organs of the entity model, and the observation information includes an input of a piercing bullet wound penetrating the organ, an input of a pain associated with a source part of an arbitrary disease, or an input of a pathological change.

10. The method according to claim 5, wherein the internal components existing inside the entity model include a plurality of biological tissues, and the observation information includes a brush input associated with at least two or more biological tissues among the plurality of biological tissues.

11. A method of providing an observation information sharing service by a service server, the method comprising the steps of:
receiving observation information input from a plurality of user terminals; and
searching for an association pattern on an entity from the received observation information.

12. A computer-readable storage medium storing instructions for performing a method of providing a service for inputting and sharing observation information on an arbitrary entity, wherein
the method of providing a service for inputting and sharing observation information on an arbitrary entity includes the steps of:
loading a three-dimensional entity model;
providing an interface capable of inputting the observation information onto the three-dimensional entity model; and
receiving the observation information from a user through the interface, wherein
the observation information includes a painting input by the user using a marker displayed on the entity model.
